(19) United States
(12) Patent Application Publication
(10) Pub. No.: US 2013/0029870 A1
(43) Pub. Date: Jan. 31, 2013

(54) MATERIALS AND METHODS FOR DIAGNOSIS OF ASTHMA

(71) Applicants: SHYAM S. MOHAPATRA, Lutz, FL (US); John J. Lima, Jacksonville, FL (US)

(72) Inventors: SHYAM S. MOHAPATRA, Lutz, FL (US); John J. Lima, Jacksonville, FL (US)

(73) Assignees: THE NEMOURS FOUNDATION, JACKSONVILLE, FL (US); UNIVERSITY OF SOUTH FLORIDA, TAMPA, FL (US)

(21) Appl. No.: 13/604,467

(22) Filed: Sep. 5, 2012

Related U.S. Application Data
(63) Continuation of application No. 12/663,820, filed on Jun. 11, 2010.
(60) Provisional application No. 60/945,473, filed on Jun. 21, 2007.

(51) Int. Cl. C12Q 1/68; C40B 30/04

(52) U.S. Cl. 506/9; 435/6.11

(57) ABSTRACT

The present invention pertains to materials and methods for diagnosing and/or determining the prognosis and likelihood of developing asthma. In one embodiment, methods of the invention comprise the use of single nucleotide polymorphic markers of asthma. Markers of the invention are present in the atria natriuretic peptide (NPPA) gene, and serve as a marker for genetic susceptibility of a person or animal in developing asthma.

### CROSS-REFERENCE TO RELATED APPLICATIONS

This application is a continuation of U.S. application Ser. No. 12/663,820, filed Jun. 11, 2010, now abandoned, which is the National Stage of International Application Number PCT/US2008/067920, filed Jun. 23, 2008, which claims the benefit of U.S. Provisional Application Ser. No. 60/945,473, filed Jun. 21, 2007, the disclosures of which are hereby incorporated by reference in their entirety, including all figures, tables and amino acid or nucleic acid sequences.

### GOVERNMENT SUPPORT

This invention was made with Government support under grants awarded by the National Institute of Health (NIH), grant numbers R03DK57734, R01HL071394, and R01HL074755. The Government has certain rights in the invention.

### BACKGROUND OF THE INVENTION

Asthma is a chronic complex disease characterized by inflammation, constriction of the airways, and bronchial hyper-responsiveness to external stimuli. Asthma affects an estimated 20 million people in the United States and 300 million people worldwide. Asthma is the most common childhood disease and is the third leading cause of hospitalization among individuals under 18 years of age. African Americans are three to four times more likely than Caucasians to be hospitalized for asthma, and are four to six times more likely to die from asthma. Numerous candidate genes have been linked to asthma, and susceptibility loci for asthma have been mapped to regions in most chromosomes (Palmer et al., 2001; Cookson, 2002; Haagerup et al., 2002; Malerba et al. 2005; Park, 2006; Bosse et al., 2007; Holloway et al., 2007). Moreover, a region on chromosome 1p36 has been identified as an asthma candidate loci (Haagerup et al., 2002).

Natriuretic peptides (NP) consist of a family of peptides that are synthesized mainly in the atria and ventricles (Costello, 2006) and are distributed in most tissues of the body (Pandey, 2005). NP regulate renal hemodynamics, vascular cell function, cell growth, cell proliferation, cardiovascular function, and reproductive cell function. The NP family includes three homologous peptides: atrial natriuretic peptide (ANP), B-type natriuretic peptide (BNP), and C-type natriuretic peptide (CNP), which are products of 3 different genes (Pandey, 2005). The gene encoding the synthesis of ANP preprohormone, NPPA, consists of 3 exons separated by 2 introns, and is located on the long arm of chromosome 1 (p36.2). Exon 3 encodes the synthesis ANP, which consists of 28 amino acids (Vesely, 2001).

ANP has been localized in various tissue of the lung suggesting that the lung synthesizes the peptide (Mohapatra, 2004). Moreover, the ANP system plays an important role in the biological activities of the lung including vasodilation, bronchorelaxation, pulmonary permeability, and surfactant production and action (Sakamoto et al., 1986; Perreault et al., 1995). ANP is also thought to play an important role in augmenting allergic inflammation and in the genesis of asthma (Mohapatra, 2004).

Natriuretic peptides (NP) are a family of peptides that include ANP, BNP, and CNP, and are distributed in most tissues of the body (Pandey, 2005), including the lung (Mohapatra, 2004; Hamad et al., 2003). ANP relaxes human airway epithelium and smooth muscle, induces bronchodilation and is bronchoprotective against several different stimuli in subjects with asthma (Chanez et al., 1990; Angus et al., 1993; Hulks et al., 1991; Hulks et al., 1994; Angus et al., 1994; Angus et al., 1995). ANP is also thought to play an important role in augmenting allergic inflammation and in the genesis of asthma (Mohapatra, 2004). ANP evokes its effects in the lung by activating the natriuretic peptide receptor A, a particulate transmembrane guanylyl cyclase, which catalyzes the conversion of GTP to cGMP (Pandey, 2005; Hamad et al., 2003).

The gene encoding the synthesis of ANP preprohormone, NPPA, consists of 3 exons separated by 2 introns, and is located on the long arm of chromosome 1 (p36.2). Exon 3 encodes the synthesis ANP, which consists of 28 amino acids (Vesely, 2001). Several studies have reported associations between polymorphisms in the NPPA gene with cardiovascular disease or with diabetes (Kato et al., 2000; Rubattu et al., 2004; Rubattu, 2006; Nannipieri, et al., 2003; Nannipieri et al., 2001; Roussel et al., 2004; Guo et al., 2005). A region of chromosome 1p36, which includes the NPPA gene, has been linked to asthma (Haagerup, 2002). This is the first time associations between asthma and polymorphisms in the NPPA gene have been reported.

### BRIEF SUMMARY OF THE INVENTION

The present invention pertains to identification of single nucleotide polymorphic markers of asthma. These markers are present in the atria natriuretic peptide (NPPA) gene, and serve as a marker for genetic susceptibility and are useful in diagnosis of asthma. In an exemplified embodiment, the SNP is the C allele of rs5067. In another exemplified embodiment, the SNP is the C allele of rs5065. In a further embodiment, the SNP is the A allele of rs5063.

### BRIEF DESCRIPTION OF THE DRAWING

FIGS. 1A-B are graphs demonstrating the linkage disequilibrium (LD) between NPPA SNPs rs5063, rs5065, and rs5067 in Caucasians (FIG. 1A) and African Americans (FIG. 1B) with asthma. The numbers in the block refer to values of D′, which estimates the degree of LD. Blocks with zero indicate no LD, values of 100 indicate complete LD.

### BRIEF DESCRIPTION OF THE TABLES

Table 1 is a table listing the sequences of the primers and probes used to determine the SNP genotypes of NPPA.

Table 2 is a table listing characteristics of the screening cohort.

Table 3 is a table listing characteristics of the replicate cohort.

Table 4 is a table comparing the genotype frequencies of 3 NPPA SNPs between African Americans and Caucasians.

Table 5 is a table demonstrating the influence of NPPA SNPs on the risk of asthma risk for Caucasian participants in the screening cohort.

Table 6 is a table demonstrating the influence of NPPA SNPs on the risk of asthma risk for African American participants in the screening cohort.

Table 7 is a table showing the distribution of common haplotypes of NPPA SNPs rs5063, rs5065 and rs5067 in 508 cases and 269 controls.

Table 8 is a table showing the influence of genotype for NPPA SNPs on the risk of asthma for participants in the replicate cohort.

### BRIEF DESCRIPTION OF THE SEQUENCES

SEQ ID NO:1 is a forward primer for PCR that can be used according to the subject invention.

SEQ ID NO:2 is a reverse primer for PCR that can be used according to the subject invention.

SEQ ID NO:3 is an oligonucleotide probe that can be used according to the subject invention.

SEQ ID NO:4 is a forward primer for PCR that can be used according to the subject invention.

SEQ ID NO:5 is a reverse primer for PCR that can be used according to the subject invention.

SEQ ID NO:6 is an oligonucleotide probe that can be used according to the subject invention.

SEQ ID NO:7 is a forward primer for PCR that can be used according to the subject invention.

SEQ ID NO:8 is a reverse primer for PCR that can be used according to the subject invention.

SEQ ID NO:9 is an oligonucleotide probe that can be used according to the subject invention.

SEQ ID NO:10 is a forward primer for PCR that can be used according to the subject invention.

SEQ ID NO:11 is a reverse primer for PCR that can be used according to the subject invention.

SEQ ID NO:12 is an oligonucleotide probe that can be used according to the subject invention.

SEQ ID NO:13 is a nucleotide sequence encoding a human ANP protein.

SEQ ID NO:14 is an amino acid sequence of a human ANP protein encoded by the polynucleotide of SEQ ID NO:13.

SEQ ID NO:15 is a polynucleotide encoding a human ANP protein having a SNP (rs5065) of the invention.

SEQ ID NO:16 is an amino acid sequence of a human ANP protein encoded by a polynucleotide of SEQ ID NO:15.

SEQ ID NO:17 is a polynucleotide encoding a human ANP protein having a SNP (rs5063) of the invention.

SEQ ID NO:18 is an amino acid sequence of a human ANP protein encoded by a polynucleotide of SEQ ID NO:17.

### DETAILED DISCLOSURE OF THE INVENTION

The subject invention concerns materials and methods for detecting, diagnosing, and/or determining the prognosis or likelihood of developing asthma in a person or animal. In one embodiment, the presence or absence of a single nucleotide polymorphism (SNP) in a gene encoding a natriuretic peptide is used for diagnosis of asthma or determining prognosis or likelihood of developing asthma. In one embodiment, the gene is the NPPA gene. In an exemplified embodiment, the SNP is the C allele of rs5067. In another exemplified embodiment, the SNP is the C allele of rs5065. In a further embodiment, the SNP is the A allele of rs5063.

Three SNPs in the NPPA gene were associated with asthma in both African American and Caucasian participants in the screening cohort:
G/A (rs5063) in exon 1 resulting in the substitution of a methionine (ATG) in place of the valine (GTG) at position 32 in the human ANP sequence;
T/C (rs5065) in exon 3 resulting in the substitution of an arginine (CGA) in place of the termination codon (TGA) at position 152 in the human ANP sequence;
T/C (rs5067) in the 3′ untranslated region of the human ANP sequence.
   The minor alleles for rs5065 and rs5067 were associated with a reduced the risk of asthma by 50% to 55% in Caucasians and by 62 to 64% in African Americans when the odd ratios were adjusted for age, gender and BMI. The minor allele for rs5063 was associated with reduced risk of asthma by 57% and 64% in Caucasians and African Americans, respectively, but the p-values (≈0.02) just missed statistical significance according to a cutoff value of 0.0167. For Caucasians, the association was validated between SNP rs5067 and asthma: the adjusted odd ratio was 0.24, indicating that the A-allele reduced the risk of asthma by 78%. Therefore, NPPA is an important susceptibility gene for asthma. It was also found that the rs5065 and rs5067 are in LD and that the GIG was the most prevalent haplotype in Caucasians (79%) and African Americans.

Methods of the invention comprise determining whether a person or animal has one or more SNPs in a gene encoding an NP, such as the NPPA gene, wherein the one or more SNPs are associated with asthma or are associated with an increased risk for developing asthma. Methods of the invention can employ any suitable method for detecting, identifying, or determining the presence or absence of one or more target SNPs. In one embodiment, one or more SNPs are identified in DNA or RNA (e.g., mRNA) or other nucleic acid, such as cDNA prepared from a DNA or RNA sample, of a person or animal. The DNA or RNA sample can be obtained, for example, from a blood, tissue, or other biological sample from the person or animal.

Those SNPs that result in a change in the amino acid sequence of an NP protein, such as ANP protein, can be detected by identifying the change in the amino acid sequence of the NP protein in a sample from the person or animal. In one embodiment, the change in amino acid sequence is determined by sequencing the protein or a fragment thereof. In another embodiment, the change in amino acid sequence is determined using an antibody that binds specifically to an ANP protein having the sequence alteration or using an antibody that binds specifically to an ANP protein that does not have the sequence alteration. Antibodies useful in the subject invention may be commercially available or can be prepared using standard methods known in the art. In an exemplified embodiment, the ANP protein is encoded by a gene having the rs5065 SNP wherein the termination codon coded for at position 152 of the amino acid sequence of wild-type ANP is changed to a codon coding for an arginine, which thereby results in a protein having an extra two arginine residues at the carboxy terminus of the protein. Specific embodiments of the rs5065 SNP are shown in SEQ ID NOs:15 and 16. In another exemplified embodiment, the ANP protein is encoded by a gene having the rs5063 SNP wherein the codon coding for valine at position 32 of the amino acid sequence of wild-type ANP is changed to a codon coding for a methionine. Specific embodiments of the rs5063 SNP are shown in SEQ ID NOs:17 and 18.

In an exemplified embodiment, one or more SNPs are detected or determined by obtaining a genetic sample from the person or animal and analyzing the sample for the presence of the one or more SNPs by melting curves of fluorescent-labelled oligonucleotide probes bound to polymerase chain reaction (PCR)-generated targets from the sample. In another embodiment, one or more SNPs are determined by sequencing a target gene sequence of the person or animal. Numerous methods for sequencing gene or polynucleotide sequences are known in the art and all are contemplated by the subject invention.

In another embodiment, one or more SNPs are detected or determined by detecting changes in endonuclease restriction sites from those sites present or absent in the wild type gene. In one embodiment, the rs5065 SNP can be detected by screening the NPPA gene for inactivation of a wild-type recognition site for the restriction endonuclease Sca I. An Sca I restriction fragment length polymorphism (RFLP) assay for the rs5065 SNP is described in Ramasawmy et al. (1992).

Methods that can be used to detect a SNP of the invention also include, but are not limited to, amplifying nucleic acid from a person or animal using an allele-specific primer capable of amplifying the target allele. The target allele sequence can be detected, for example, using an oligonucleotide probe specific for the target allele. Primers and probes of the invention can comprise from about 10 to about 50 nucleotides (i.e., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50), and typically from about 15 to about 30 nucleotides. The primers and/or probes may comprise a sequence that corresponds to or is complementary with the allele-specific sequence. The primers and probes of the invention may optionally comprise a detectable label, such as a fluorophore, an enzyme or enzyme substrate, a radioactive element or compound, etc. In one embodiment for detecting the C allele of rs5067, a primer pair of oligonucleotides comprising the sequence of SEQ ID NO:10 and SEQ ID NO:11, and/or the oligonucleotide probe comprising the sequence of SEQ ID NO:12 can be used. In one embodiment for detecting the C allele of rs5065, a primer pair of oligonucleotides comprising the sequence of SEQ ID NO:7 and SEQ ID NO:8, and/or the oligonucleotide probe comprising the sequence of SEQ ID NO:9 can be used.

Additional methods for detection of SNPs are described in U.S. Pat. No. 7,189,512, some of which are summarized herein. For example, hybridization techniques can also be used to detect or determine a SNP. An example of a suitable hybridization technique involves the use of DNA chips (oligonucleotide arrays), for example, those available from Affymetrix, Inc. Santa Clara, Calif. For details on the use of DNA chips for the detection of for example, SNPs, see U.S. Pat. No. 6,300,063 and U.S. Pat. No. 5,837,832.

SNPs in genomic DNA can also be detected using the Invader technology available from Third Wave Technologies, Inc., Madison, Wis. Messner et al. 2000; Hall et al., 2000). Two DNA probes hybridize to a target nucleic acid to form a structure recognized by a nuclease enzyme. If one of the probes is complementary to the sequence, the nuclease will cleave it to release a short DNA fragment termed a “flap”. The flap binds to a fluorescently-labeled probe and forms another structure recognized by a nuclease enzyme. When the enzyme cleaves the labeled probe, the probe emits a detectable fluorescence signal thereby indicating which SNP variant is present.

Another technique for the detection of SNPs is the Taqman assay (see, e.g., Arnold et al., 1998). A target DNA containing a SNP is amplified in the presence of a probe molecule that hybridizes to the SNP site. The probe molecule contains both a fluorescent reporter-labeled nucleotide at the 5′-end and a quencher-labeled nucleotide at the 3′-end. The probe sequence is selected so that the nucleotide in the probe that aligns with the SNP site in the target DNA is as near as possible to the center of the probe to maximize the difference in melting temperature between the correct match probe and the mismatch probe. As the PCR reaction is conducted, the correct match probe hybridizes to the SNP site in the target DNA and is digested by the Taq polymerase used in the PCR assay. This digestion results in physically separating the fluorescent labeled nucleotide from the quencher with a concomitant increase in fluorescence. The mismatch probe does not remain hybridized during the elongation portion of the PCR reaction and is, therefore, not digested and the fluorescently labeled nucleotide remains quenched.

Denaturing HPLC using a polystyrene-divinylbenzene reverse phase column and an ion-pairing mobile phase also can be used to identify SNPs. In this process, a DNA segment containing a SNP is PCR amplified. After amplification, the PCR product is denatured by heating and mixed with a second denatured PCR product with a known nucleotide at the SNP position. The PCR products are annealed and are analyzed by HPLC at elevated temperature. The temperature is chosen to denature duplex molecules that are mismatched at the SNP location but not to denature those that are perfect matches. Under these conditions, heteroduplex molecules typically elute before homoduplex molecules. For an example of the use of this technique see Kota et al. (2001).

Other techniques for SNP detection are the Single Strand Conformation Polymorphisms (SSCP) technique and the Denaturing Gradient Gel Electrophoresis (DGGE) technique. In SSCP, sample and control DNAs are denatured and run on polyacrylamide gels in a non-denaturing environment. Single strands of DNA (typically less than about 300 bp in size) with a SNP are separated on the gel, and will show different mobility as compared to the single strands of the control DNA. The mobility difference is caused by a conformational change of the single stranded DNA due to the single base change. The DGGE method is similar to SSCP in the sense that it depends on DNA denaturation by gel, but DGGE uses heat or chemical denaturants to separate the two strands of the DNA being examined. DNA fragments have different melting temperatures, determined by their nucleotide sequence. The hydrogen bonds formed between G/C melt at a higher temperature than those of T/A. When separated by electrophoresis through a gradient of increasing temperature, the control DNA and the sample DNA that contains different nucleotides will melt at different specific points on the gel, according to their melting temperatures.

In the Single Base Primer Extension assay, double stranded sample DNA is denatured and primers complementary to the sequence are added and allowed to anneal to the DNA. The primers are usually about 20 to 30 nucleotides in length, and their 3 end is adjacent to the SNP. Next, DNA polymerase and ddNTPs with varying fluorescent tags are added. By identifying the 3° base added to the primer, it is possible to identify if a SNP is present. As an alternative to using varying tags, a mass spectrometer may be employed.

Allele Specific Oligonucleotide Ligation is yet another technique employing specific primers. One primer is complementary to the target sequence 5′ to and including the SNP position. The second primer is complementary to the sequence immediately 3′ of the SNP position. The sample DNA is denatured and allowed to hybridize with the primers. DNA ligase is then added. If the upstream primer matches the SNP, ligation will be achieved between the two primers. If there is a mismatch, then the primer does not match the SNP and ligation is not achieved. Thus, if ligation has taken place, the product will be a single strand with the two primers connected together.

Another assay for SNPs is Allele Specific Hybridization. As with Allele Specific Oligonucleotide Ligation, sample DNA is examined by hybridization to primers. In this technique, an oligonucleotide fabricated onto a solid support covers the SNP and regions 5° and 3′ of the SNP. Sample DNA is denatured and allowed to hybridize to the oligonucleotide/solid support. The sample DNA/oligonucleotide/solid support is analyzed by eluting the bound sample DNA. When the SNP position in the sample DNA is complementary to the base located at the same position on the fabricated oligonucleotide, the two strands are separated with more difficulty than when there is a mismatch. This technique can be used in conjunction with various labels.

In another embodiment, one or more SNPs in a nucleic acid can be detected using a sequence-specific ribozyme (U.S. Pat. Nos. 5,591,610; 6,025,167; 6,180,399; and 6,696,250). Target alleles can be distinguished from non-target alleles using a sequence-specific ribozyme to digest the nucleic acid wherein sequences that have the target sequence of the ribozyme can be distinguished from sequences that do not have the target sequence using, for example, differences in melting temperature of the sequences or cleavage digestion assays.

In another embodiment, one or more SNPs in a nucleic acid can be detected using ligase chain reaction (LSR), single strand polymorphism assays (SSPA), or heteroduplex analysis assays (Orita et al., 1989).

The subject invention also concerns methods for treating a person or animal in need of treatment, comprising i) detecting one or more SNPs in a gene encoding an NP, such as the NPPA gene, wherein the one or more SNPs are associated with asthma or an increased risk for asthma, and ii) administering an effective amount of a drug or compound for treating asthma and/or taking steps to limit the likelihood of an asthma attack from occurring. In one embodiment, the SNP is the C allele of rs5067 and/or the C allele of rs5065 and/or the A allele of rs5063.

The subject invention also concerns kits for the detection of one or more SNPs associated with asthma or associated with an increased risk for developing asthma. In one embodiment, a kit of the invention comprises, in one or more separate containers, one or more oligonucleotide primers and/or one or more oligonucleotide probes specific for a SNP of the invention. In one embodiment, a kit comprises any one or more of an oligonucleotide forward primer comprising the nucleotide sequence of SEQ ID NO:1, SEQ ID NO:4, SEQ ID NO:7, or SEQ ID NO:10. In one embodiment, a kit comprises any one or more of an oligonucleotide reverse primer comprising the nucleotide sequence of SEQ ID NO:2, SEQ ID NO:5, SEQ ID NO:8, or SEQ ID NO:11. In one embodiment, a kit comprises any one or more of an oligonucleotide probe comprising the nucleotide sequence in SEQ ID NO:3, SEQ ID NO:6, SEQ ID NO:9, or SEQ ID NO:12. The oligonucleotide probe of the kit can optionally be labeled with a detectable moiety or the kit can optionally contain a detectable moiety that can be used to label an oligonucleotide probe. In another embodiment, a kit of the invention can contain a one or more components for detecting changes in endonuclease restriction sites in a wild-type gene encoding an NP, such as the NPPA gene. In one embodiment, the kit comprises a Sca I restriction endonuclease. Kits of the invention can also optionally comprise suitable polymerases, buffers (e.g., PBS), and/or nucleotides. Kits of the invention can also optionally contain packaging information and/or instructions for use of the kit reagents in a method of the invention. Containers in a kit of the invention can be composed of any suitable material, such as glass or plastic.

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequence includes both the sense and antisense strands either as individual strands or in the duplex.

The methods and compositions of the present invention can be used in the diagnosis and detection of asthma in humans and other animals. The other animals contemplated within the scope of the invention include domesticated, agricultural, or zoo- or circus-maintained animals. Domesticated animals include, for example, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses.

All patents, patent applications, provisional applications, and publications referred to or cited herein are incorporated by reference in their entirety, including all figures, tables, nucleic acid sequences, amino acid sequences, and drawings, to the extent they are not inconsistent with the explicit teachings of this specification.

### Materials and Methods

Selection of Cases and Controls.

Individuals with well-characterized asthma were selected from two different clinical trials that were sponsored by the American Lung Association Asthma Clinical Research Centers. The first trial, Safety of Inactivated Influenza Vaccination in Asthma (SIIVA), studied over 2000 children and adults with asthma (ALA, ACRC, 2001); the second trial, Effectiveness of Low Dose Theophylline as Add-On Therapy in the Treatment of Asthma (LODO), studied 489 individuals with poorly controlled asthma (ALA, ACRC, 2007). The screening cohort consisted of 336 Caucasian and 143 African American participants from SIIVA (Lima et al., 2006), and 154 Caucasian and 74 African American healthy, non-asthmatic controls. The replicate cohort consisted of 172 Caucasian participants from LODO (ALA, ACRC, 2007) and 115 healthy controls that were recruited as a genetic control population. An African American replicate cohort was not studied owing to an insufficient number of African Americans in the replicate healthy control group. Control subjects were healthy by physical examination, denied having asthma, a history of asthma, or asthma symptoms, and were not taking any asthma drugs or other drugs chronically. All participants were between the ages of 3 and 75 years old. Representatives of other racial groups were not studied owing to the small number of subjects.

Genotyping and Determination of Haplotypes.

Four SNPs in the NPPA gene were typed: C/G (rs13305986) in the promoter; G/A (rs5063) in Exon 1 resulting in Met32→Val substitution; T/C (rs5065) in Exon 3 resulting in an Arg152→Ter substitution; and T/C in the 3′UT region (rs5067). These were selected because in previous studies they were associated with cardiovascular diseases or diabetes (Kato et al., 2000; Rubattu et al., 2004; Rubattu, 2006; Nannipieri, et al., 2003; Nannipieri et al., 2001; Roussel et al., 2004; Guo et al., 2005), or the variant was common. Rs5065 SNP in Exon 3 has been associated with diabetic nephropathy (Roussel et al., 2004), stroke (Rubattu et al., 2004) and insulin resistance (Guo et al., 2005). The promoter polymorphism rs13305986 has been associated with increased left ventricular mass index (Rubattu et al., 2004) and hypertension (Kato et al., 2000). Rs5067 is one of five known SNPs in the 3′ UT that is common with a heterozygosity of 0.23. Other SNPs in the 3′ UT are infrequent (heterozygosity <0.1) or their frequencies are undetermined.

The SNP genotypes of NPPA were determined by analyzing the melting curves of fluorescent-labeled oligo probes bound to PCR-generated targets from individual DNA samples on a LightTyper instrument (Roche Applied Science, Indianapolis, Ind.). The sequences of the primers (Operon Techniologies, Alameda, Calif.) and probes (Roche) are listed in the Table 1.

The PCR cocktail consists of 20 mM Tris-HCl (pH 8.4), 50 mM KCl, 3 mM MgCl₂, 0.2 mM dNTPs, 1 μM probe-targeted strand primer, 0.1 μM non-targeted strand primer, 0.2 μM Probe, 0.5 U JumpStart Taq DNA Polymerase (Sigma, St. Louis, Mo.), and 50 ng DNA in a total volume of 20 μl overlaid with 20 μl mineral oil. The PCR conditions are 60 cycles of denaturation at 95° C. for 30 seconds, annealing at 55° C. for 30 seconds, and polymerization at 72° C. for 20 seconds using a DNA Thermal 9600 (Perkin Elmer Cetus, Oak Brook, Ill.), followed by 5 minutes at 95° C. and 10 minutes at 50° C. The 96-well PCR plate is then transferred to the LightTyper instrument for melting curve analysis following the manufacturer's instruction.

Violation of the assumption of Hardy-Weinberg Equilibrium (HWE) among the SNPs was examined using a classical χ² goodness-of-fit test by SAS JMP 6. Linkage disequilibrium analyses were performed by Haploview 3.32. Haplotypes were constructed using HAP (Halperin et al., 2004). Descriptive statistics were utilized to assess subjects' demographic information and distributions of allele, genotype and haplotype. Genetic additive and dominant effects were considered in the genotypic model. Fisher's exact test was used to examine the significance of the association between two variables in a 2×2 contingency table with small sample size. If any genetic association with asthma was found in the preliminary χ² analysis, Cochran's and Mantel-Haenszel statistics were further estimated including crude odds ratio (OR) and association hypothesis test. Because possible confounding factors including age, gender and body mass index (BMI) could bias the results, adjusted OR were also calculated by logistic regression, implementing these factors as covariates into the model (Balding et al., 2001). The population-attributable risk (PAR) was determined using standard techniques. A Bonferroni's factor was used to define the threshold of significance, which was 0.0167 (0.05/3 SNPs). One SNP was not in HWE and therefore was not studied further.

Following are examples which illustrate procedures for practicing the invention. These examples should not be construed as limiting. All percentages are by weight and all solvent mixture proportions are by volume unless otherwise noted.

### EXAMPLES

### Example 1

### Baseline Characteristics

Characteristics of the screening cohort are shown in Table 2. The mean ages among the 4 groups (Caucasian cases and controls and African American cases and controls) are in reasonable agreement. The percent of participants that were female from SIIVA tended to be slightly lower (64%) compared to Caucasian controls (73%), African American participants from SIIVA (76%) and controls (80%). The Body Mass Indexes (BMI) and prevalence of obesity in African American cases and controls were higher compared to Caucasian cases and controls. Asthma drug use among asthmatic participants was evenly distributed among Caucasians and African Americans; about 50% were on daily-inhaled corticosteroids. Asthma severity was greater among African Americans compared to Caucasian participants as judged by lower values for peak flow, FEV1 and % predicted FEV1. The scores for asthma symptom utility index (ASUI) indicate that SIIVA participants had mild asthma.

Characteristics of the replicate cohort are shown in Table 3. Age and gender for the Caucasian LODO and control participants were reasonably matched. However, the BMI and prevalence of obesity in LODO participants and in controls were different. Compared to Caucasian participants in SIIVA, participants in LODO were older, a higher proportion were on inhaled steroids, and had more severe asthma as judged by lower ASUI scores and lower pulmonary function measurements. Owing to the small number of African American participants, particularly in the replicate control cohort, associations between NPPA SNPs and asthma were not analyzed.

### Example 2

### Genotyping

A high rate of genotyping was achieved for the four SNPs (>91%). For both African American and Caucasian controls, rs13305986 was not in HWE (p=<0.0001, p=0.0003), and was no longer considered in the analyses. The minor allele frequency for rs13305986 was 2.4%, which was in excellent agreement with the frequency of 2.6% in a European population of hypertensives reported by Rubattu et al. (2006), who reported HWE for this SNP. The reason for this difference is not clear. The frequencies of CC homozygotes in cases and controls were similar, 94.2% and 95.2%, respectively, ruling out asthma as a factor contributing to not achieving HWE for this SNP. Therefore, rs13305986 was no longer considered in the analyses. The remaining SNPs were in HWE for both African American and Caucasian controls, except for rs5063 in African Americans, which may have been related to small numbers. Genotype frequencies differed by race (see Table 4). Frequencies of the minor alleles for rs5063 A, rs5065 C, and rs5067 A were significantly higher in African Americans compared to Caucasians: 0.074, 0.369, and 0.381 vs. 0.045, 0.160, and 0.100, respectively (p<0.002).

### Example 3

### Determination of Linkage Disequilibrium and Haplotypes

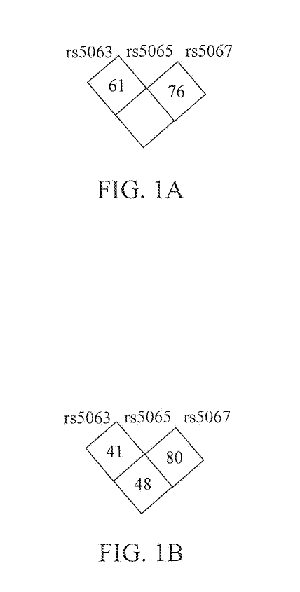

Strong pair-wise linkage disequilibrium (LD) was observed between rs5065 and rs5067 (D′ was 0.76; r²=0.33 P<0.0001). Six haplotypes were identified (Table 7), with GTT, GCC and GCT being the most common (45%). No differences in haplotype frequencies were observed between cases in the screening cohort and in the replicate cohort. The r² values for the LD between rs5065 and rs5067 in African American and Caucasians were 0.4 and 0.63, respectively (p<0.0001). Out of the 9 possible haplotypes, 7 were found in African Americans and 6 in Caucasians. GTG and GCA were the most common haplotypes in African Americans and Caucasians, together comprising 86% of the total (FIG. 1).

### Example 4

### Association Analyses in Screening Cohorts

For both African Americans and Caucasians, significant associations were found between asthma and rs5065 and rs5067, with a trend for rs5063 (see Tables 5 and 6). Owing to the small number of minor homozygotes and heterozygotes for rs5063, rs5065 and rs5067, a dominant model was assumed in both Caucasians and African Americans. For Caucasians, the risk of asthma was found to be reduced by 57% and 50% respectively, in carriers of the C-allele for rs5065 and in carriers of the A-allele for rs5067 (see Table 5). For African Americans, we found that the risk of asthma was reduced by 64% and 62% respectively, in carriers of the C-allele for rs5065 and in carriers of the A-allele for rs5067 (see Table 6). For both African Americans and Caucasians, carriers of the A-allele of rs5063 were associated with a reduced risk of asthma, however the p-values for the adjusted odds ratio (p=0.019 and 0.02) missed the cut-off p-value of 0.0167. Analyses of asthma risks in both racial groups by allele frequencies for the 3 SNTs were also significant.

The GTG haplotype was associated with asthma in both African Americans and Caucasians (see Table 3).

### Example 5

### Association Analyses in Replicate Cohorts

Owing to the small number of minor homozygotes and heterozygotes for rs5063, rs5065 and rs5067, we assumed a dominant model. Significant associations were found between asthma and rs5065 and rs5067 in the screening cohort, with a trend for rs5063 (Table 5). The adjusted OR [95% confidence interval (CI)] in carriers of the C allele for rs5065 was 0.45 (0.29-0.70; P<0.0001), indicating that the risk of asthma was reduced by 55% compared with TT homozygotes in the screening cohort. The adjusted OR (95% CI) for carriers of the C allele for rs5067 was 0.5 (0.29-0.84; P=0.009), indicating that that the risk of asthma was reduced by 50% compared with TT homozygotes. To validate our findings for Caucasians in the screening cohort, we analyzed associations between asthma and rs5063, rs5065, and rs5067 SNPs in our replicate cohort (Table 5). The risk of asthma was reduced by 75% in carriers of the C-allele for rs5067, thereby replicating the results for this SNP in the screening cohort. In contrast, we did not replicate our finding for rs5063 and rs5065 SNP associations with asthma. Small numbers of participants in our replicate controls (N=4) and cases (N=66) prevented replicate analyses in African Americans. However, the minor allele frequencies for rs5063, rs5065, and rs5067 in African American participants from LODO (N=66) were 0.038, 0.386, and 0.45, respectively, which were in reasonable agreement with allele frequencies in our African American asthmatic cases in our screening cohort (Table 6) except for rs5067. The minor allele frequency for this SNP was close to the corresponding allele frequency in African American controls in our screening cohort.

To control for the possible confounding effect of the clinical trials, a conditional association test was performed to estimate the effect of the rs5067 SNP over the screening and replicate cohorts using the Cochran-Mantel-Haenszel statistic with the Breslow-Day test for homogeneity of the ORs (http://v8doc.sas.com/sashtml/stat/chap28/sect39.htm).

After adjusting for the trial, we found an association between asthma and rs5067 (P<0.0001). Carriers of the C allele had a decreased risk of asthma compared with controls (OR=0.47; 95% CI 0.31-0.60). The large P-value for the Breslow-Day test (0.96) indicates no significant trial difference in the ORs between the two trials.

The PAR for carriers of the C allele for rs5067 was 23.3%, which suggests that in this population, if all participants carried the C allele, one would estimate a reduction of 23% in the prevalence of asthma.

In the screening cohort, 11% of asthmatic cases carried one or two copies of the GCC haplotype compared with 20.8% in controls: adjusted OR (95% CI)=0.52 (0.30-87); P=0.013. For the GCT haplotype, 10.4% of the cases carried one or two copies compared with 18.8% in controls: adjusted OR (95% CI)=0.47 (0.27-0.82); P=0.008. We were not able to replicate these associations. It should be noted that our sample size is underpowered to evaluate comprehensively the associations between haplotype of the NPPA gene and asthma.

### Example 6

Two SNPs in the NPPA gene were associated with asthma in the screening cohort: T/C (rs5065) in Exon 3 resulting in an Arg152→Ter substitution; and T/C in the 3′UT region (rs5067). The C allele for rs5065 and the C allele for rs5067 were associated with a reduced risk of asthma by 50-55%. The association between SNP rs5067 and asthma was validated in the replicate cohort. Carriers of the C allele in cases and controls for the screening cohorts (0.136 vs. 0.25) were very similar to the frequencies we observed in the replicate cohort (0.134 vs. 0.25), suggesting that the association between rs5067 and asthma is real. For participants in the screening cohort and in the replicate cohort, the C allele reduced the risk of asthma 50 (95% CI 0.29-0.84) to 76% (95% CI 0.11-0.53) (Tables 5 and 8). This is the first report of an association between asthma and a genetic variant in the NPPA gene. Data herein complement data in studies of human alveolar and bronchial epithelial cells, and in murine models that ANPs play an important role in lung physiology and in asthma (Mohapatra et al., 2004; Sakamoto et al., 1986; Perreault et al., 1995; Kumar et al., 2002; Hellermann et al., 2004). The results herein are also consistent with a genetic study reporting that a region on chromosome 1p36, which harbours the NPPA gene, has been identified as an asthma candidate locus (Haagerup et al., 2002). Thus, NPPA is an important susceptibility gene for asthma.

The rs5065 and rs5067 are in LD and the GTT was the most prevalent haplotype in Whites, followed by the GCC and GCT haplotypes. The GCC and OCT haplotypes were significantly associated with a reduced risk of asthma in the screening cohort. However, these findings were not replicated, indicating that the 3-SNP haplotype is no more informative than genotype of the rs5067 SNP.

It is not clear how the rs5067 SNP affects the function of the NPPA gene and the encoded protein, and ultimately the mechanism underlying its role in the pathogenesis of asthma. A survey by the MicroInspector program computationally reveals that there are many miRNA target sites in NPPA mRNA (NM-006172). SNP rs5067 in the 3′-UTR of NPPA mRNA locates within the target sites of microRNAs hsamiR-205 and hsa-miR-328. Change of nucleotide T to C of rs5067 results in the loss of hsa-miR-205 binding and in the creation of hsa-miR-328 binding. Thus, it is possible that the highly frequent SNP rs5067 may play a major role in regulating ANP activity. It is also possible that the rs5067 SNP is in LD with one or more SNPs that regulate transcription or translation of message encoding the ANP and/or the function of the protein. For example, rs5067 is in tight LD with several SNPs in the CLCN6 (location 1p36) gene and less so in the MTHFR (location 1p36.22) gene, which are adjacent to NPPA.

Results herein show that the C allele of rs5067 is associated with an adjusted OR of 0.5-0.25 in Whites, which represents a modest reduction in the risk of asthma. However, given that the frequency of rs5067 is common, the PAR was 23%, which estimates the fraction of cases of disease that would be avoided if exposure (genotype) were removed (Hunter et al., 2007).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

TABLE 1

| SNP ID | Forward primer | Reverse primer | Probe |
|---|---|---|---|
| rs13305986 | cacgaagagtctgagca | ggtcaggcgaaagga | ttgCtatttctatttgggggttggattcctgg- |
| | (SEQ ID NO: 1) | (SEQ ID NO: 2) | SPC-Fluorescein |
| | | | (SEQ ID NO: 3) |
| rs5063 | agacgtaggccaagag | ggagtgagcacagcat | TggacaCggcattgtacatgggat- |
| | (SEQ ID NO: 4) | (SEQ ID NO: 5) | SPC-Fluorescein |
| | | | (SEQ ID NO: 6) |
| rs5065 | cactcatacatgaagctgac | tctacagttagcataagatgtg | AtgtctgtgttctctttgcagtacCgaag- |
| | (SEQ ID NO: 7) | (SEQ ID NO: 8) | SPC-Fluorescein |
| | | | (SEQ ID NO: 9) |
| rs5067 | cactcatacatgaagctgac | tctacagttagcataagatgtg | AcaCaaatgcagcagagaccccagg- |
| | (SEQ ID NO: 10) | (SEQ ID NO: 11) | SPC-fluorescein |
| | | | (SEQ ID NO: 12) |

TABLE 2

| Characteristic, | Self-Identified Ethnicity | | | |
|---|---|---|---|---|
| mean ± SE or | Whites | | African Americans | |
| Percent | SIIVA | Control | SIIVA | Control |
| Number | 336 | 154 | 143 | 74 |
| Age, years | 35 ± 1 | 29.6 ± 0.65 | 32 ± 2 | 30.1 ± 1.0 |
| Female, % | 64.1 | 73.4 | 75.5 | 79.7 |
| Obese, % | 30.9 | 34.4 | 49.7 | 61.0 |
| Current smokers, % | 3.3 | 0 | 7.7 | 0 |
| Former smokers, % | 21.1 | 0 | 11.9 | 0 |
| Daily inhaled steroid, % | 51.8 | 0 | 53.9 | 0 |
| Daily long-acting beta agonist, % | 28.1 | 0 | 25.4 | 0 |
| Daily leukotriene modifier, % | 33.9 | 0 | 29.4 | 0 |
| ASUI | 0.85 ± 0.01 | NA | 0.80 ± 0.01 | NA |
| Peak Flow, L/min | 397 ± 7.0 | NA | 322 ± 10 | NA |
| FEV1, L | 2.69 ± 0.05 | NA | 2.00 ± 0.07 | NA |
| % predicted FEV1 | 87.1 ± 1.1 | NA | 83.2 ± 2.0 | NA |

TABLE 3

| Characteristic, | Self-Identified Ethnicity | | | |
|---|---|---|---|---|
| mean ± SE or | Whites | | African Americans | |
| Percent | LODO | Control | LODO | |
| Number | 154 | 116 | 74 | |
| Age, years | 30 ± 0.65 | 30.6 ± 1.2 | 30 ± 1 | |
| Female, % | 70.2 | 59.6 | 79.7 | |
| Obese, % | 32.9 | 8.6 | 60.8 | |
| Current smokers, % | 0 | 0 | 0 | |
| Former smokers, % | 22.4 | 0 | 14.1 | |
| Daily inhaled steroid, % | 82.0 | 0 | 68.9 | |
| Daily long-acting beta | 21.7 | 0 | 23.4 | |
| agonist, % | | | | |
| Daily leukotriene | 4.35 | 0 | 1.6 | |
| modifier, % | | | | |
| ASUI | 0.68 ± 0.01 | NA | 0.71 ± 0.02 | NA |
| Peak Flow, L/min | 371 ± 8.1 | NA | 355 ± 15 | NA |
| FEV1, L | 2.55 ± 0.06 | NA | 2.22 ± 0.06 | NA |
| % predicted FEV1 | 78.3 ± 1.3 | NA | 76.6 ± 2.3 | NA |

TABLE 4

| | | White | Black | |
|---|---|---|---|---|
| | | Frequency | Frequency | P value for Chi-Square- |
| SNP | Genotype | N (%) | N (%) | test |
| Rs5063 | AA | 2 (0.3) | 7 (2.7) | 0.002 |
| | AG | 61 (8.4) | 24 (9.3) | |
| | GG | 661 (91.3) | 226 (87.9) | |
| Rs5065 | CC | 21 (2.9) | 33 (13.1) | <0.0001 |
| | TC | 186 (26.1) | 120 (47.6) | |
| | TT | 507 (71.0) | 99 (39.3) | |
| rs5067 | AA | 17 (2.3) | 37 (14.7) | <0.0001 |
| | AG | 111 (15.3) | 118 (46.8) | |
| | GG | 598 (82.4) | 97 (38.5) | |

TABLE 5

| | | Whites | | | | |
|---|---|---|---|---|---|---|
| | | Cases | Controls | | Adjusted Odds | |
| SNP | Genotype | N (%) | N (%) | Odds | ratio (95% CI) | p value |
| rs5063 | AG | 18 (6.1) | 19 (12.8) | 0.95 | 0.43 (0.21-0.88) | 0.02 |
| | GG | 275 (93.9) | 129 (87.2) | 2.13 | 1 | |
| rs5065 | CC + TC | 70 (24.1) | 60 (41.4) | 1.17 | 0.45 (0.29-0.70) | <0.0001 |
| | TT | 220 (75.9) | 85 (58.6) | 2.59 | 1 | |
| rs5067 | CC + TC | 40 (13.6) | 37 (25.0) | 1.08 | 0.50 (0.29-0.84) | 0.009 |
| | TT | 254 (66.5) | 111 (75.0) | 2.29 | 1 | |

TABLE 6

| | | Blacks | | | | |
|---|---|---|---|---|---|---|
| | | Cases | Controls | | Adjusted Odds | |
| SNP | Genotype | N (%) | N (%) | Odds | ratio (95% CI) | P value |
| rs5063 | AA + AG | 11 (9.6) | 16 (21.9) | 0.69 | 0.36 (0.15-0.85) | 0.019 |
| | GG | 103 (90.4) | 57 (78.1) | 1.81 | 1 | |
| rs5065 | CC + TC | 57 (50.9) | 53 (74.6) | 1.08 | 0.36 (0.18-0.70) | 0.003 |
| | TT | 55 (49.1) | 18 (25.4) | 3.06 | 1 | |
| rs5067 | AA + AG | 54 (49.1) | 52 (72.2) | 1.04 | 0.38 (0.20-0.74) | 0.004 |
| | GG | 56 50.9) | 20 (27.8) | 2.8 | 1 | |

TABLE 7

| | | | Cumulative |
|---|---|---|---|
| Haplotype number | Haplotype | Frequency (%) | frequency (%) |
| 1 | GTT | 79.2 | 79.2 |
| 2 | GCC | 7.7 | 86.9 |
| 3 | GCT | 7.2 | 94.1 |
| 4 | ATT | 4.1 | 98.2 |
| 5 | GTC | 1.7 | 99.9 |
| 6 | ACT | 0.1 | 100 |

TABLE 8

| | | Participants | | | |
|---|---|---|---|---|---|
| | Geno- | Cases | Control | Adjusted Odds | |
| SNP | type | N (%) | N (%) | Ratio (95% CI)* | P-value |
| rs5063 | AA + | 17 (9.9) | 9 (8.1) | 1.02 (0.36-2.83) | 0.98 |
| | AG | | | | |
| | GG | 155 (90.1) | 102 (91.9) | 1 | |
| rs5065 | CC + | 53 (30.8) | 24 (22.4) | 1.16 (0.60-2.24) | 0.66 |
| | TC | | | | |
| | TT | 119 (69.2) | 83 (77.6) | 1 | |
| rs5067 | CC + | 23 (13.4) | 28 (25.0) | 0.24 (0.11-0.53) | <0.0001 |
| | TC | | | | |
| | TT | 149 (86.6) | 84 (75.0) | 1 | |

*Adjusted for age, gender and body mass index.
CI, confidence interval.

### REFERENCES

U.S. Pat. No. 5,591,610
U.S. Pat. No. 5,837,832
U.S. Pat. No. 6,025,167
U.S. Pat. No. 6,180,399
U.S. Pat. No. 6,300,063
U.S. Pat. No. 6,696,250
U.S. Pat. No. 7,189,512
ALA, ACRC. (2001) “The safety of inactivated influenza vaccine in adults and children with asthma” N Engl J Med 345:1529-36.
ALA, ACRC. (2007) “Clinical trial of low-dose theophylline and montelukast in patients with poorly controlled asthma” Am J Respir Crit Care Med 175:235 12.
Angus, R. M. et al. (1993) “Bronchodilator, cardiovascular, and cyclic guanylyl monophosphate response to high-dose infused atrial natriuretic peptide in asthma” Am Rev Respir Dis 147:1122-5.
Angus, R. M. et al. (1994) “Effect of inhaled atrial natriuretic peptide on methacholine induced bronchoconstriction in asthma” Clin Exp Allergy 24:784-8.
Angus, R. M. et al. (1995) “Effect of inhaled atrial natriuretic peptide and a neutral endopeptidase inhibitor on histamine-induced bronchoconstriction” Am J Respir Crit Care Med 151:2003-5.
Arnold et al., (1998) BioTechniques 25(1):98 106.
Balding D, Bishop M. Handbook of statistical genetis. Hoboken, N.J.: Wiley, 2001,
Bosse, Y. et al. (2007) “Toward a comprehensive set of asthma susceptibility genes” Annu Rev Med 58:171-84.
Chanez, P. et al. (1990) “Atrial natriuretic factor (ANF) is a potent bronchodilator in asthma” J Allergy Clin Immunol 86:321-4.
Cookson, W. O. (2002) “Asthma genetics” Chest 121:7S-13S.
Costello, J. M. et al. (2006) “A review of the natriuretic hormone system's diagnostic and therapeutic potential in critically ill children” Pediatr Crit Care Med 7:308-18.
Guo, X. et al. (2005) “Hypertension genes are genetic markers for insulin sensitivity and resistance” Hypertension 45:799-803.
Haagerup, A. et al. (2002) “Asthma and atopy—a total genome scan for susceptibility genes” Allergy 57:680-6.
Hall et al., (2000) PNAS 97(15):8272-77.
Halperin E, Eskin E. Haplotype reconstruction from genotype data using imperfect phylogeny. Bioinformatics 2004; 20:1842-9.
Hamad, A. M. et al. (2003) “Guanylyl cyclases, nitric oxide, natriuretic peptides, and airway smooth muscle function” Am J Physiol Lung Cell Mol Physiol 285:L973-83.
Hellermann, G. et al. (2004) “Mechanism of bronchoprotective effects of a novel natriuretic hormone peptide” J Allergy Clin Immunol 113:79-85.
Hessner et al., (2000) Clinical Chemistry 46(8):1051-56.
Holloway, J. W. et al. (2007) “Identifying novel genes contributing to asthma pathogenesis” Curr Opin Allergy Clin Immunol 7:69-74.
Hulks, G. et al. (1991) “Influence of elevated plasma levels of atrial natriuretic factor on bronchial reactivity in asthma” Am Rev Respir Dis 143:778-82.
Hulks, G. et al. (1994) “High dose inhaled atrial natriuretic peptide is a bronchodilator in asthmatic subjects” Eur Respir J 7:1593-7.
Hunter, D. J. et al. (2007) “Drinking from the fire hose—statistical issues in Genomewide Association Studies” N Engl J Med 357:436-9.
Kato, N. et al. (2000) “Genetic analysis of the atrial natriuretic peptide gene in essential hypertension” Clin Sci (London) 98:251-8.
Kota et al., (2001) Genome 44(4):523-28.
Kumar, M. et al. (2002) “Atrial natriuretic peptide gene transfer by means of intranasal administration attenuates airway reactivity in a mouse model of allergic sensitization” J Allergy Clin Immunol 110:879-82.
Lima J J, Feng H, Duckworth L et al. (2007) “Association analyses of adrenergic receptor polymorphisms with obesity and metabolic alterations” Metabolism; 56:757-65.
Lima, J. J. et al. (2006) “The C523A beta2 adrenergic receptor polymorphism associates with markers of asthma severity in African Americans” J Asthma 43:185-91.
Malerba, G. et al. (2005) “A review of asthma genetics: gene expression studies and recent candidates” J Appl Genet 46: 93-104,
Mohapatra, S. S. (2004) “Lockey R F, Vesely D L, Gower W R Jr. Natriuretic peptides and genesis of asthma: an emerging paradigm?” J Allergy Clin Immunol 114:520-6.
Nannipieri, M. et al. (2001) “Polymorphisms in the hANP (human atrial natriuretic peptide) gene, albuminuria, and hypertension” Hypertension 37:1416-22.
Nannipieri, M. et al. (2003) “Association between polymorphisms of the atrial natriuretic peptide gene and proteinuria: a population-based study” Diabetologia 46:429-32.
Orita et al. (1989) Proc. Natl. Acad. Sci. 86:2766.
Palmer, L. J. et al. (2001) “Using single nucleotide polymorphisms as a means to understanding the pathophysiology of asthma” Respir Res 2:102-12.
Pandey, K. N. (2005) “Internalization and trafficking of guanylyl cyclase/natriuretic peptide receptor-A” Peptides 26:985-1000.
Park, H. S. (2006) “The role of novel genes in modifying airway responses in asthma” Curr Allergy Asthma Rep 6:112-6.
Perreault, T. et al. (1995) “Role of atrial natriuretic factor in lung physiology and pathology” Am J Respir Crit Care Med 151:226-42.
Ramasawmy et al. (1992) Hum genet. 90:323-324.
Revicki D A, Leidy N K, Brennan-Diemer F, Sorensen S, Togias A. Integrating patient preferences into health outcomes assessment the multiattribute Asthma Symptom Utility Index. Chest 1998; 114:998-1007.
Roussel, R. et al. (2004) “Investigation of the human ANP gene in type I diabetic nephropathy: case-control and follow-up studies” Diabetes 53:1394-8.
Rubattu, S. et al. (2004) “Atrial natriuretic peptide gene polymorphisms and risk of ischemic stroke in humans” Stroke 35:814-8.
Rubattu, S. et al. (2006) “Association of atrial natriuretic peptide and type a natriuretic peptide receptor gene polymorphisms with left ventricular mass in human essential hypertension” J Am Coll Cardiol 48:499-505.
Sakamoto, M. et al. (1986) “The lung as a possible target organ for atrial natriuretic polypeptide secreted from the heart” Biochem Biophys Res Commun 135:515-20.
Vesely, D. L. (2001) “Atrial natriuretic peptides in pathophysiological diseases” Cardiovasc Res 51:647-58.

We claim:

1. A method for diagnosing asthma or determining genetic susceptibility to asthma in a person comprising the steps of obtaining a nucleic acid sample from the person, and detecting in nucleic acid of the person a single nucleotide polymorphic (SNP) marker in a NPPA gene encoding atrial natriuretic peptide (ANP), and associating the presence or absence of said SNP marker with a diagnosis of asthma or with susceptibility to asthma in the person, wherein said SNP marker is detected by detecting in said gene a nucleotide change associated with said SNP marker using a nucleic acid amplification assay or a nucleic acid sequencing method.

2. The method according to claim 1, wherein said SNP marker detected is the SNP designated as rs5067.

3. The method according to claim 1, wherein said SNP marker detected is the SNP designated as rs5065.

4. The method according to claim 1, wherein said SNP marker is detected by a nucleic acid amplification assay.

5. The method according to claim 1, wherein said SNP marker is detected by a nucleic acid sequencing method.

6. The method according to claim 1, wherein said nucleic acid is DNA.

7. The method according to claim 1, wherein said SNP marker is detected using an oligonucleotide primer pair, wherein said primer pair comprises the nucleotide sequence of SEQ ID NOs:1 and 2, SEQ ID NOs:4 and 5, SEQ ID NOs:7 and 8, or SEQ ID NOs:10 and 11.

8. The method according to claim 1, wherein said SNP marker is detected using an oligonucleotide probe, wherein said probe comprises the nucleotide sequence of SEQ ID NO:3, SEQ ID NO:6, SEQ ID NO:9, or SEQ ID NO:12.

9. The method according to claim 1, wherein two or more of said SNP markers are detected.

10. The method according to claim 3, wherein said rs5065 SNP marker results in the substitution of an arginine codon in place of a termination codon at position 152 in the human ANP sequence.

11. The method according to claim 3, wherein said rs5065 SNP marker results in the codon at position 152 in human ANP sequence being changed from TGA to CGA.

12. The method according to claim 3, wherein human ANP gene comprising said rs5065 SNP marker comprises the nucleotide sequence of SEQ ID NO:15.

13. The method according to claim 3, wherein human ANP encoded by a human ANP gene comprising said rs5065 SNP comprises the amino acid sequence of SEQ ID NO:16.

14. The method according to claim 1, wherein said SNP marker detected is the SNP designated as rs5063.

15. A method for detecting a single nucleotide polymorphic (SNP) marker in a NPPA gene encoding atrial natriuretic peptide (ANP) of a person or animal, wherein said method comprises detecting in the NPPA gene of the person or animal a SNP marker using a nucleic acid amplification assay or a nucleic acid sequencing method, wherein the SNP is one designated as rs5067, rs5065, or rs5063.


SEQUENCE LISTING (21 sequences)

SEQ ID NO: 1 - DNA, 17 bp, Artificial Sequence
  forward primer for PCR
cacgaagagtctgagca

SEQ ID NO: 2 - DNA, 15 bp, Artificial Sequence
  reverse primer for PCR
ggtcaggcgaaagga

SEQ ID NO: 3 - DNA, 32 bp, Artificial Sequence
  oligonucleotide probe
ttgctatttctatttgggggttggattcctgg

SEQ ID NO: 4 - DNA, 16 bp, Artificial Sequence
  forward primer for PCR
agacgtaggccaagag

SEQ ID NO: 5 - DNA, 16 bp, Artificial Sequence
  reverse primer for PCR
ggagtgagcacagcat

SEQ ID NO: 6 - DNA, 24 bp, Artificial Sequence
  oligonucleotide probe
tggacacggcattgtacatgggat

SEQ ID NO: 7 - DNA, 20 bp, Artificial Sequence
  forward primer for PCR
cactcatacatgaagctgac

SEQ ID NO: 8 - DNA, 22 bp, Artificial Sequence
  reverse primer for PCR
tctacagttagcataagatgtg

SEQ ID NO: 9 - DNA, 29 bp, Artificial Sequence
  oligonucleotide probe
atgtctgtgttctctttgcagtaccgaag

SEQ ID NO: 10 - DNA, 20 bp, Artificial Sequence
  forward primer for PCR
cactcatacatgaagctgac

SEQ ID NO: 11 - DNA, 22 bp, Artificial Sequence
  reverse primer for PCR
tctacagttagcataagatgtg

SEQ ID NO: 12 - DNA, 25 bp, Artificial Sequence
  oligonucleotide probe
acacaaatgcagcagagaccccagg

SEQ ID NO: 13 - DNA, 840 bp, Homo sapiens
  CDS (95)..(547)
agggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccggtactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggac

SEQ ID NO: 14 - protein, 151 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 15 - protein, 151 aa, Homo sapiens; identical to SEQ ID NO: 14

SEQ ID NO: 16 - DNA, 840 bp, Homo sapiens
  CDS (95)..(553)
agggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccggtaccgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggac

SEQ ID NO: 17 - protein, 153 aa, Homo sapiens [sequence omitted]

SEQ ID NO: 18 - protein, 153 aa, Homo sapiens [sequence omitted]

SEQ ID NO: 19 - DNA, 840 bp, Homo sapiens
  CDS (95)..(547)
agggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccatgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccggtactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggac

SEQ ID NO: 20 - protein, 151 aa, Homo sapiens
MSSFSTTTVSFLLLLAFQLLGQTRANPMYNAMSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 21 - protein, 151 aa, Homo sapiens; identical to SEQ ID NO: 20